(19) United States
(12) Patent Application Publication
(10) Pub. No.: US 2020/0305820 A1
(43) Pub. Date: Oct. 1, 2020

(54) DIAGNOSTIC AND THERAPEUTIC UNIT

(71) Applicant: ISTITUTI FISIOTERAPICI OSPITALIERI (IFO)-ISTITUTO REGINA PER LO STUDIO E LA CURA DEI TUMORI (IRE), Rome (RM) (IT)

(72) Inventors: Urbano Barbabella, Roma (RM) (IT); Antonella Soriani, Roma (RM) (IT); Giuseppe Iaccarino, Roma (RM) (IT); Valeria Landoni, Roma (RM) (IT); Lidia Strigari, Roma (RM) (IT); Marcello Banassi, Roma (RM) (IT); Concetta Ronsivalle, Roma (RM) (IT); Luigi Picardi, Roma (RM) (IT)

(21) Appl. No.: 16/089,602

(22) PCT Filed: Apr. 8, 2016
(86) PCT No.: PCT/IB2016/052002

§ 371 Date: Sep. 28, 2018

(51) Int. Cl. A61B 6/00; A61B 6/04; A61N 5/10

(52) U.S. Cl. CPC A61B 6/502; A61B 6/0435; A61N 2005/1094; A61N 5/1045; A61N 5/1077; A61N 5/1031

(57) ABSTRACT

A medical apparatus, particularly suitable for the treatment of a breast cancer, which apparatus has:
a support for a patient, such as to expose a breast in a predetermined position of the support;
an imaging unit, configured in such a way as to provide, in use, a 3D image of the breast;
a treatment unit, having delivery means of a ionizing radiation beam and configured so as to provide, in use, the beam at the breast;
a control unit of the imaging unit and/or treatment unit, configured to determine the delivery of a radiation beam with a shape dependent upon the image of the target body district supplied by the imaging unit.

### TECHNICAL FIELD OF THE INVENTION

The present invention relates to a diagnostic and therapeutic unit, suitable for the management of patients suffering from breast cancer.

### BACKGROUND

Over the past two decades, the development of radio diagnostic (imaging) and radiotherapy techniques have allowed huge advances in the diagnosis and treatment of cancer, particularly of breast cancer that is, alone, about 30% of all cancer cases.

In particular, there are early diagnostic systems based on ultrasound, mammography, computed tomography (CT) and/or Magnetic Resonance (MR). Similarly, radiotherapy and radiosurgery systems increasingly sophisticated have emerged.

Diagnosis and post-operative therapy of breast cancer are now being addressed in a low synergic way.

In fact, the diagnostic step is first performed, which consists in a targeted investigation conducted in a dedicated laboratory using the above mentioned imaging techniques; where this diagnostic phase highlights a pathology, surgery and radiotherapy treatment follows, the latter being usually done in several sessions, with high-energy linear accelerators installed in rooms specially shielded for protection purposes (bunkers). Unfortunately, due to the long waiting lists the treatment can be considerably delayed compared to surgery which precedes it, with detriment to the effectiveness of that treatment.

In general, the goal of radiation therapy with external beams is to maximize the dose delivered to the target (i.e. the site of the tumour before surgery), while minimizing the irradiation of surrounding healthy tissue.

Some radiotherapy techniques provide for the positioning of the patient in the prone position, with the purpose to further displace organs at risk from the target allowing in this way also to deliver the therapeutic dose in a smaller number of fractions (hypo fractionation) with evident benefits in terms of commitment of the patient and the lender structure.

### SUMMARY OF THE INVENTION

The technical problem posed and solved by the present invention is therefore to provide a diagnostic and treatment apparatus which allows to obviate the drawbacks mentioned above with reference to prior art.

This problem is solved by an apparatus according to claim 1.

Preferred features of the present invention are object of the dependent claims.

The invention realizes an integration between an imaging instrument and a radiotherapy instrument, the latter in particular based on a linear particle accelerator, LINAC, preferably designed in order to be able to rotate about the axis of the breast and having an energy optimal for the particular application (typically not more than 3 MeV). Preferably, the radiotherapy instrument is self-shielding for use in environments not particularly shielded.

This integration allows the imaging instrument to provide the necessary data for treatment planning, taking into account the actual morphology of the organ after the surgical removal of the malignancy and the position assumed by the organ during the treatment.

Moreover, before the dispensing step the imaging instrument can be used for the positioning verification.

By virtue of the possibility to carry out the treatment of the breast in the prone position and the careful planning and verification allowed, the apparatus of the invention allows to increase the dose per fraction (hypo-fractionation), thereby reducing by more than 30% the number of sessions required, with undeniable benefits of costs for healthcare organizations and patients' quality of life.

Preferably, the apparatus provides a treatment bed ergonomically designed and shielded for the protection of the patient, with the possibility of prone treatment of the right breast and of the left breast indifferently.

Furthermore, the apparatus may provide for the integration of all components of the system on a single base, of low size and weight, such as to be placed in a minimum size laboratory (e.g. 4×4 meters), without the need for interventions of floor reinforcement.

The apparatus of the invention lends itself to be employed at any health facility, even if not provided with a bunker for conventional radiotherapy.

Other advantages, features and the modes of employ of the present invention will become apparent from the following detailed description of some embodiments thereof, given by way of example and not for limitation.

### BRIEF DESCRIPTION OF THE DRAWINGS

Reference will be made to the figures of the accompanying drawings, in which:

FIG. 1 shows an example block diagram of an apparatus according to a preferred embodiment of the present invention;

FIG. 2 shows a schematic perspective view of the apparatus of FIG. 1;

FIG. 2A shows a perspective view of a main movable screen of the apparatus of FIG. 2, while in FIG. 2B is shown a secondary shield for protecting the patient.

### DETAILED DESCRIPTION OF PREFERRED EMBODIMENTS

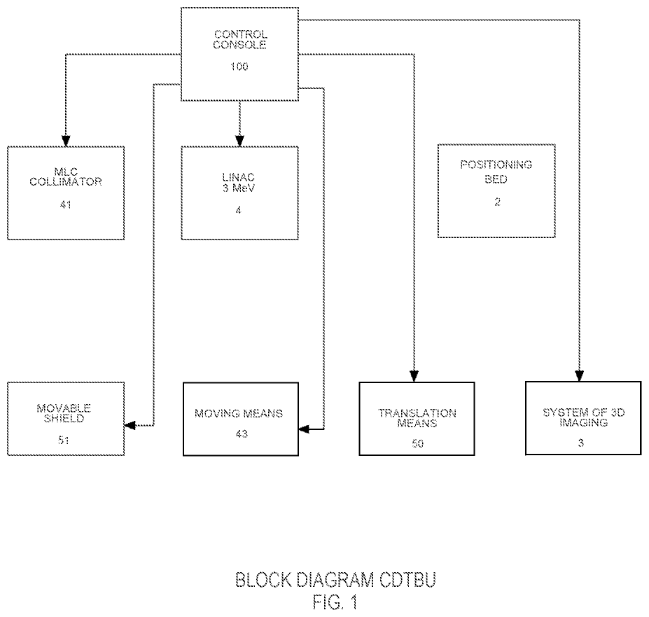

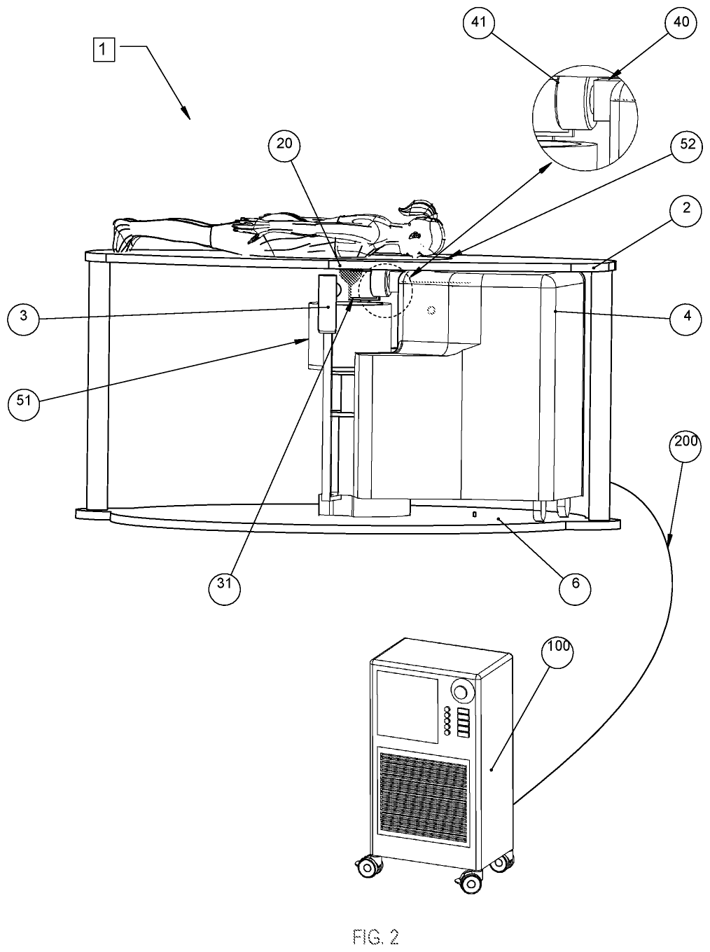

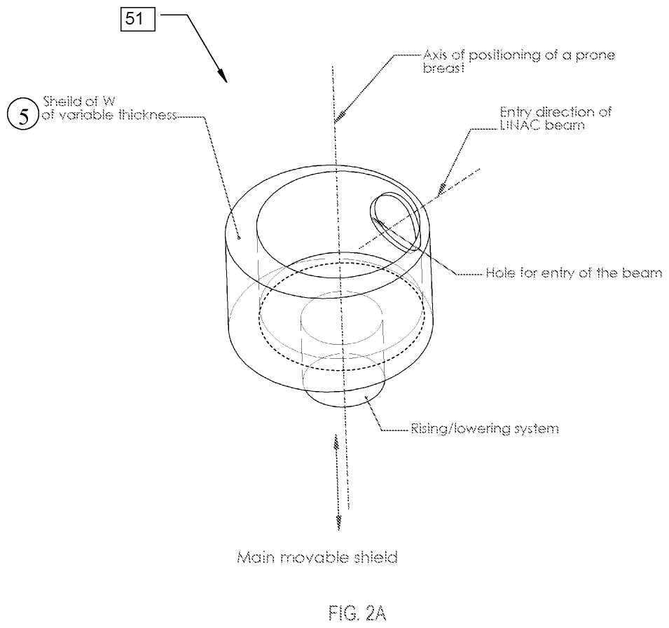

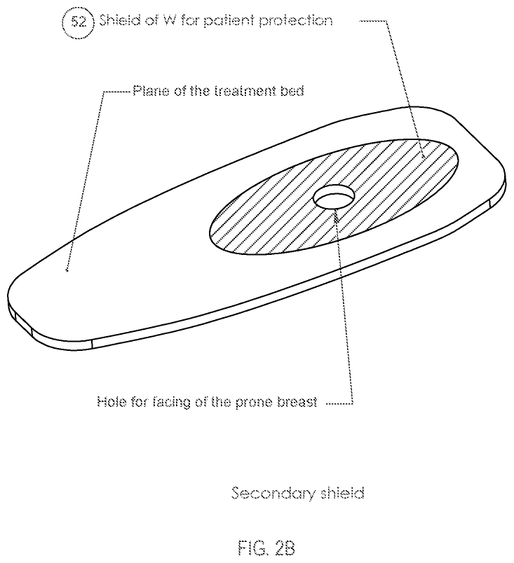

With reference to FIGS. 1 and 2, a medical apparatus according to a preferred embodiment of the invention is generally denoted by 1.

The medical apparatus 1 is an apparatus particularly suitable for the treatment of a breast cancer.

The apparatus 1 mainly comprises a support 2 for a patient, an imaging unit 3, a treatment unit 4, a control unit 100 of said imaging and treatment unit 3, 4 and a connecting cable 200 between the two units.

The support 2 is configured in such a way as to allow the exposure of a target body district, for example a breast, in a predetermined position 20 suitable for the treatment. In a preferred embodiment of the invention, the support 2 comprises a bed suitable to accommodate a patient in the prone position. Furthermore, this bed includes, at the predetermined position 20, also an opening apt to allow the exposure of a breast towards the treatment unit 4 and the imaging unit 3. Said opening may be accompanied by special adapters to be used depending on the size of the treated breast.

In the embodiment in which the bed with the opening as described above is present, the positioning of the patient on the support 2 in the prone position allows the correct exposure of the breast with respect to the treatment and imaging unit 4, 3, while ensuring the exclusion of areas of the body not involved in the treatment.

The imaging unit 3, preferably essentially consisting of a low energy X-ray source (50-80 KV), a solid-state detection system, an electronic management and a dedicated software, is configured in such a way to provide, in use, a three dimensional image of the target body district.

The treatment unit 4 mainly comprises dispensing means 41 of a beam of ionizing radiation and is configured so as to dispense, in use, the beam at the target (i.e. the site of the tumour before surgery).

The treatment unit 4 can be a radiation therapy unit, in particular comprising a linear particle accelerator (LINAC).

Preferably, the accelerator is of low energy 2-4 MeV (or not more than 3 MeV) and may, moreover, have a particularly compact configuration.

In one embodiment, the treatment unit 4 comprises a main collimator 41 which realizes the above-mentioned dispensing means of the radiation beam and which is suitable to shape the same beam according to a selected profile. Preferably, the main collimator 41 is a multileaf collimator (Multi-Leaf Collimators, MLC). The MLC are described in the known prior art and generally comprise a plurality of slats of lead or tungsten, which are moved sliding in the direction perpendicular to the radiation beam to be detected. In this way, the slats define a hole of shape and dimensions corresponding to those of the target anatomical structure for the passage of the beam. In a particular embodiment, the MLC may be of the type with rotating sectors, in particular such as that described in patent application n. IT RM2014A000431, which due to its dimensional characteristics is more suitable to be inserted in the structure of the apparatus of the invention. This patent application is herein incorporated by this reference.

Furthermore, the treatment unit 4 can also comprise a primary collimator 40, arranged upstream of the main collimator 41 with respect to the radiation beam.

In a particularly advantageous embodiment, primary radiation shield means 51 and secondary radiation shield means 52 can be associated with the treatment unit 4. Preferably, the primary shield means 51 comprises a cylindroid shaped screen with a hole configured to allow the passage of the radiation beam delivered from the treatment unit 4 toward the target body district. Preferably, the screen has wall of thicknesses variable depending on the intensity of the beam of radiation to be shielded.

In a preferred embodiment, the primary radiation shield means 51 are movable, in particular selectively displaceable between a shielding position and a resting position. In the shielding position the shield means 51 is interposed between the treatment unit 4 and the support 2 of the patient in such a way as to shield body portions adjacent to the target body district from the radiation beam, in addition to reducing the dose in the neighborhood of the treatment room to levels compatible with a safety use of the apparatus without any particular shields to the same room, thereby configuring the equipment as “self-shielded”.

In a preferred embodiment, the movable shield means 51 are translatable so that, preferably, the shielding position is a raised position and the resting position is a lowered position.

The possibility of movement of the shield means 51 above described allows an operator to access the target body district for its correct positioning when said means are in a lowered position or in any case in a resting position.

Furthermore, this possibility of lowering the screen will also be used in the phase of radiographic imaging of the breast, when the source, placed at 90° to the direction of the beam of the accelerator, will have to reach the detector opposed to it.

Finally, actuating means for the movable shield means 51 can also be provided, such as, for example, a motor. Such means, translation means in this example, is denoted by 50 in FIG. 1.

The secondary shield means 52 can be incorporated in the plane of the treatment bed, in the neighborhood of the hole for the positioning of the breast, and has the function to protect the patient from radiation scattered radially to the direction of the treatment beam. In a preferred embodiment, such secondary means comprises a screen preferably having a predominantly elliptical shape, as shown in FIG. 2B.

In a preferred embodiment, the treatment unit 4 and/or the imaging unit 3 are movable with respect to the support 2. Preferably, the movement is effected according to a predetermined path around the predetermined position 20 provided for the target body district. In particular, the predetermined path can be a substantially circular path, for example, centered at the predetermined position 20. Preferably, the treatment unit 4 and/or the imaging unit 3 are movable below the support 2.

Then, in an exemplifying manner, the treatment unit 4 and/or imaging unit 3 can be capable of traveling in the two verses an entire circumference by moving below the support 2, in particular with center on the axis of the positioning hole 20 of the target body district.

Advantageously, the shield means are integral with the treatment unit 4 and/or imaging unit 3 with respect to the movement of the latter.

In FIG. 1 the moving means 43 are represented in a schematic way, precisely associated with the treatment unit 4 and the imaging unit 3.

In the apparatus shown by way of example in FIG. 2, the imaging unit and the treatment unit are configured in such a way that the respective beams of imaging and treatment are delivered in a direction substantially respectively orthogonal to one another.

The apparatus of the invention can also comprise a common base 6 of the support 2, the treatment unit 4 and the imaging unit 3 (and also the shield means 5). On this base 6 can be formed guides for the movement of the units 3 and 4 according to the said predetermined path.

Regarding the control unit 100 of the imaging unit 3 and treatment unit 4, it is configured to manage the delivery of a radiation beam shaped in function of the image of the target body district supplied from said imaging unit 3. For this purpose, the control unit 100 acts on the dispensing means 41.

In a preferred embodiment, the control unit 100 is configured in such a way to process, preferably in real time, a treatment plan of the treatment unit 4 as a function of the data supplied from the imaging unit 3.

In general terms, the control unit 100 can be configured for a coordinated control of the imaging unit 3, the treatment unit 4, preferably of the collimator of the latter, and/or of the shield means 51. In exemplifying and non-limiting manner, the control unit 100 can include a software of management and control of treatment unit 4 and/or imaging unit 3, a software for capturing radiographic images, a software for processing of the treatment plan and/or a software for the management of the main collimator.

The present invention has hereto been described with reference to preferred embodiments. It is to be understood that there may be other embodiments afferent to the same inventive core, as defined by the scope of protection of the claims set out below.

1. A medical apparatus configured for the treatment of a breast tumor, which medical apparatus comprises:
a support of a patient, configured to expose a target body district in a predetermined position of the support;
an imaging unit, configured in such a way as to provide an image of the target body district in said predetermined position;
a treatment unit, comprising a delivery system of a ionizing radiation beam and configured so as to provide said ionizing radiation beam at the target body district in said predetermined position;
a control unit of said imaging unit or treatment unit, configured to determine the delivery of a radiation beam with a shape dependent upon an image of the target body district supplied by said imaging unit;
a common base of said support, treatment unit and imaging unit, which common base has guides for a movement of said treatment unit and imaging unit with respect to said support according to a predetermined path around said predetermined position of the target body district,
wherein said support comprises a bed configured for receiving a patient in the prone position and bearing, at said predetermined position, an opening configured to allow the exposure of a breast towards said treatment unit or said imaging unit,
and wherein said medical apparatus further comprises:
a radiation shield for a breast in the prone position, which radiation shield is fixed with said treatment unit with respect to the movement of the treatment unit,
which radiation shield is movable between a raised shielding position, wherein it is interposed between said treatment unit and said support in such a way as to shield portions of a patient body adjacent to the target body district from the radiation beam, and a lowered resting position,
wherein said radiation shield comprises a main body containing a lateral hole configured to allow passage of a radiation beam delivered by said treatment unit towards the target body district in said predetermined position when said radiation shield is in the raised position.

2. The medical apparatus according to claim 1, wherein said imaging unit is essentially constituted by an X-ray source of low energy, by a solid state detection system, by a managing electronics and by a dedicated software.

3. The medical apparatus according to claim 1, wherein said treatment unit is a radiotherapy unit, in particular comprising a linear particle accelerator (LINAC).

4. The medical apparatus according to claim 1, wherein said treatment unit comprises a main collimator of a delivered radiation beam, programmed to shape the delivered radiation beam according to a selected profile.

5. The medical apparatus according to claim 4, wherein said treatment unit comprises a primary collimator and wherein said main collimator is arranged downstream of said primary collimator.

6. (canceled)

7. The medical apparatus according to claim 1, wherein said predetermined path is a substantially circular path, optionally centered at said predetermined position provided for the target body district.

8. The medical apparatus according to claim 1, wherein said treatment unit or said imaging unit are movable below said support.

9. The medical apparatus according to claim 1, wherein said shield comprises a screen incorporated in said support, optionally in a neighbourhood of said predetermined position.

10. The medical apparatus according to claim 1, wherein said radiation shield is translatable.

11. The medical apparatus according to claim 1, wherein said main body of said radiation shield has walls of variable thickness.

12. The medical apparatus according to claim 1, wherein said main body of said radiation shield has a substantially cylindrical configuration.

13. The medical apparatus according to claim 1, wherein said imaging unit and said treatment unit are configured in such a way that respective imaging and treatment beams are delivered in a direction substantially orthogonal to one another.

14. (canceled)

15. The medical apparatus according to claim 1, wherein said control unit is configured to process a treatment plan of the target body district by said treatment unit as a function of data supplied from said imaging unit.

16. The medical apparatus according to claim 1, wherein said control unit is configured for a coordinated control of said imaging unit, of said treatment unit, of said main collimator of the treatment unit, or of said shield.

17. The medical apparatus according to claim 1, configured for the imaging and treatment of breast as target body district.
